(19) United States
(12) Patent Application Publication
(10) Pub. No.: US 2007/0173828 A1
(43) Pub. Date: Jul. 26, 2007

(54) Spondylolistheses correction system and method of correcting spondylolistheses

(75) Inventors: Paul John Firkins, Worcs (GB); Albert Veldhuizen, Eelde (NL)

Correspondence Address: LAHIVE & COCKFIELD, LLP, ONE POST OFFICE SQUARE, BOSTON, MA, 02109-2127, US

(73) Assignee: DePuy Spine, Inc., Raynham, MA (US)

(21) Appl. No.: 11/337,159

(22) Filed: Jan. 20, 2006

(51) Int. Cl. A61F 2/30

(52) U.S. Cl. 606/61

(57) ABSTRACT

A spinal correction element in a device and method of correcting spondylolistheses in a patient transitions between a first shape and a second shape while having a first portion fixed to a forwardly displaced vertebra and a second portion fixed to an undeformed portion of the spinal column to apply a corrective force bringing a displaced vertebra into alignment with the rest of the spinal column. A shape memory material in the spinal correction element transitions from a flexible martensitic state, in which the spinal correction element matches the shape of the deformity caused by the spondylolistheses, to a rigid austenitic state, in which the spinal correction element has the shape of an expected corrected spine, to apply a corrective force to the vertebral bodies fixed to the spinal correction element, thereby correcting the deformity caused by spondylolistheses. The shape memory material is induced to transition between the rigid austenitic state and the flexible martensitic state by changing the temperature, pressure, stress, chemistry and/or another parameter of the shape memory material.

### FIELD OF THE INVENTION

The present invention relates to spinal correction devices used in orthopedic surgery. More particularly, the present invention relates to a device and method for correcting spondylolistheses in a patient.

### BACKGROUND OF THE INVENTION

Spondylolisthesis, known as “spondy”, is a displacement disorder of the lumbar or cervical spine, in which one vertebral body is forwardly displaced over another vertebral body. Spondylolisthesis may be caused by a traumatic event or by degeneration of the spine. At times, the displacement disorder is accompanied by or caused by a fracture or partial collapse of one or more vertebrae or degeneration of a disc in the spine. Patients who suffer from such conditions can experience moderate to severe distortion of the thoracic skeletal structure, diminished ability to bear loads, loss of mobility, extreme and debilitating pain, and oftentimes suffer neurological deficits in nerve function.

Spinal correction systems may be used in orthopedic surgery to correct a deformity or misalignment caused by spondylolisthesis, as well as to stabilize and/or fix vertebral bodies in a desired relationship relative to each other. A standard surgical procedure for correcting spondylolisthesis in the current state of the art involves first distracting the vertebrae at the level that the spondylolisthesis occurs, pulling the forward-translated vertebra back into alignment with the rest of the spinal column, and then stabilizing the spine while the vertebrae are held in the aligned position using posterior spinal implants consisting of anchoring devices and rigid spinal fixation elements. An interbody fusion device may also be used to give further stability and correction of the disc height, which may be compromised during the spondylolisthesis event. Compression across the vertebrae may be applied across the construct to set the correct balance of forces in the region.

The spinal fixation element used in such spinal correction systems is generally a relatively rigid fixation rod or plate that is coupled to a bone by attaching the spinal fixation element to various anchoring devices, such as hooks, bolts, wires or screws. The spinal fixation element can extend between two bone regions to effect stabilization, positioning, reduction or fixation of the bones. The spinal fixation element can have a predetermined contour that has been designed according to the properties of the target implantation site and, once installed, the spinal fixation element holds the bones in a desired spatial relationship, either until desired healing or spinal fusion has occurred, or for some longer period of time.

Standard posterior screw systems, such as the Moss Miami, Monarch, TiMX, VSP and Expedium, available from DePuy Spine, Inc, of Raynham, Mass. have been used by surgeons to correct spondylolisthesis.

Prior surgical procedures and devices for correcting spondylolisthesis are inadequate and present several difficulties. For example, the technique of pulling the forwardly displaced vertebral body back into alignment before attaching the spinal fixation elements can be difficult, painful and inaccurate for several reasons. For example, the forces required to pull the vertebral body back into alignment can be very large and/or uneven, difficult to control and/or cause damage to the patient and/or the implants the surgeon is instrumenting with. In addition, significant force is required to hold the vertebral body in alignment during subsequent attachment of the spinal fixation elements. Specialized instruments are required to carry out the procedure, which may increase the cost of the procedure.

In addition, certain spinal structures, such as muscles and ligaments, may be resistant to correction. Over time, visco-elastic forces from these spinal structures may reduce forces holding the displaced vertebral body back in alignment, resulting in only a partial correction of the deformity. When only partial correction is achieved, the surgeon may be required to re-operate at a later date, potentially causing additional complications and increased cost.

### SUMMARY OF THE INVENTION

The present invention provides a spondylolistheses correction system including a spinal correction element, such as a spinal rod, which may be formed at least partially of a shape memory material, such as nitinol. The spinal correction element transitions between a first shape and a second shape while having a first portion fixed to a forwardly displaced vertebra and a second portion fixed to an undeformed portion of the spinal column to apply a corrective force bringing the displaced vertebra into alignment with the rest of the spinal column.

For a spinal correction element formed of a shape memory material, the shape memory material preferably transitions from a flexible martensitic state, in which the spinal correction element matches the shape of the deformity caused by the spondylolistheses, and a rigid austenitic state, in which the spinal correction element has the shape of an expected corrected spine, to apply a corrective force to the vertebral bodies fixed to the spinal correction element, thereby correcting the deformity caused by spondylolistheses. Preferably, the spinal correction element transitions between the martensitic state and the austenitic state, where the spinal correction element has a pre-selected, rigid shape, by controlling the temperature of the shape memory material. When cooled below a selected temperature, (for example, below the austenitic start temperature or the martensitic start temperature) the material becomes at least partially martensitic and exists in the relatively soft, flexible martensitic state, allowing the material to be shaped to match the spondylolistheses deformity and fixed to the spinal column. When heated above a selected temperature (preferably the austenitic final temperature), the material returns to the austenitic state, in which the shape of the material matches the expected shape of the corrected spine, bringing the displaced vertebra into alignment with the rest of the spinal column. Preferably, the material is fully austenitic in the body to apply a constant corrective force to the spinal column.

According to one aspect of the invention, a method of correcting spondylolistheses in a spinal column in a patient is provided. The method comprises the steps of anchoring a first bone anchor to a first vertebra and a second bone anchor to a second vertebra that is displaced forward of the first vertebra due to the spondylolistheses, connecting the first bone anchor and the second bone anchor using a spinal corrective element, and changing the shape of the spinal corrective element to apply a corrective force to reduce the spondylolistheses.

According to another aspect of the invention, a method of correcting spondylolistheses comprises shaping a spinal corrective element to match a deformity caused by spondylolisthesis and connecting the shaped spinal corrective element to a first vertebra and a second vertebra displaced from the first vertebra in a forward direction due to the spondylolistheses.

According to still another aspect of the invention, a method of correcting spondylolistheses comprises inserting a spinal corrective element in a rod-receiving portions connected to bone anchors, wherein the spinal correction element includes a shape memory material and is shaped to match a deformity caused by the spondylolistheses, and transitioning the shape memory material from a martensitic state to an austenitic state to correct the deformity caused by the spondylolistheses.

In still another aspect of the invention, a method of correcting spondylolistheses comprises the steps of fixing a spinal corrective element to a first vertebra and a second vertebra that is forwardly displaced relative to the first vertebra due to the spondylolistheses and applying a substantially even corrective force distributed across the vertebrae to pull the second vertebra into alignment with the first vertebra.

According to another aspect of the invention, a spondylolistheses correction system comprises a first set of bone anchors connected to a first vertebra, a second set of bone anchors connected to a second vertebra that is displaced forward of the first vertebra due to the spondylolistheses, and a spinal corrective element including a shape memory material connecting the first set of bone anchors and the second set of bone anchors.

### BRIEF DESCRIPTION OF THE FIGURES

The foregoing and other objects, features and advantages of the invention will be apparent from the following description and apparent from the accompanying drawings, in which like reference characters refer to the same parts throughout the different views. The drawings illustrate principles of the invention and, although not to scale, show relative dimensions

FIGS. 1A and 1B illustrate a spondylolistheses correction system according to one embodiment of the invention.

FIG. 2 illustrates a spondylolistheses correction system including two spinal rods according to another embodiment of the invention.

FIG. 3 is a flow chart diagramming the steps involved in correcting a deformity caused by spondylolistheses according to an illustrative embodiment of the invention.

FIG. 4 illustrates a spondylolistheses correction system and deformed spinal column prior to attachment of the spondylolistheses correction system to the spinal column according to the illustrative embodiment of the invention.

FIG. 5 illustrates a spondylolistheses correction system attached to a deformed spinal column prior to correction according to the illustrative embodiment of the invention.

FIG. 6 illustrates a spondylolistheses correction system attached to a deformed spinal column after to correction of the spondylolistheses deformity according to the illustrative embodiment of the invention.

### DETAILED DESCRIPTION OF THE INVENTION

The present invention provides an improved surgical device and method for correcting spondylolistheses in a patient that employs a shape memory material. The present invention will be described below relative to certain exemplary embodiments to provide an overall understanding of the principles of the structure, function, manufacture, and use of the instruments disclosed herein. Those skilled in the art will appreciate that the present invention may be implemented in a number of different applications and embodiments and is not specifically limited in its application to the particular embodiments depicted herein.

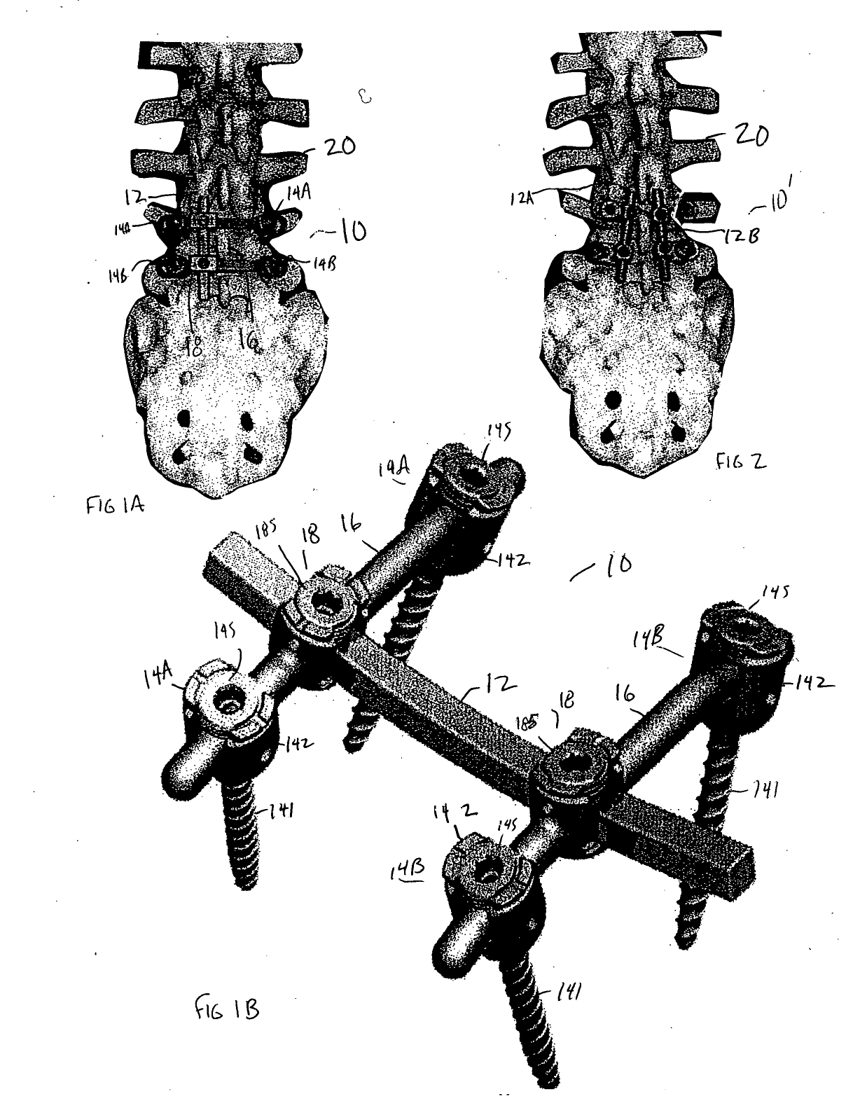

FIGS. 1A-2 illustrate an embodiment of a spondylolistheses correction system according to the teachings of the invention. As shown in FIGS. 1A and 1B, a spondylolistheses correction system 10 of an illustrative embodiment of the invention comprises at least one spinal corrective element, illustrated as a spinal rod 12, preferably formed at least partially of a shape memory material. The spondylolistheses correction system 10 of FIGS. 1A and 1B includes a single spinal rod 12, while the spondylolistheses correction system 10′ of FIG. 2 includes two spinal rods 12A and 12B straddling the vertebrae. One skilled in the art will recognize that a spondylolistheses correction system according to the teachings of the invention may have any suitable number of spinal corrective elements.

The spinal corrective element may have any suitable size and configuration for applying a corrective force to the spine, and is not limited to the illustrative rod-like configuration. The spondylolistheses correction system 10 further includes bone anchors 14, such as bone screws, for fixing the spinal rod 12 to the vertebral bodies in the spinal column 20. A first pair of bone anchors 14A, is anchored to a forwardly displace vertebra, while a second pair of bone anchors 14B is anchors to an undeformed portion of the spinal column. Several pairs of bone anchors may be used, and the invention is not limited to two pairs of bone anchors 14 as shown in FIGS. 1A-2. Transverse rods 16 connect the bone anchors 14 straddling each vertebral body to the spinal rod 12 using connectors 18.

FIG. 1B illustrates in detail the bone anchors 14, connectors 18, spinal rod 12 and transverse rods 16 used in the illustrative spondylolistheses correction system 10. As shown, each bone anchor 14 of the illustrative embodiment comprises a bone anchoring portion 141 and a rod-receiving portion 142 for receiving the transverse rod 16. A locking mechanism, such as a screw cap 145, locks the transverse rod 16 in the rod-receiving portion of the bone anchor 14. The rod-receiving portion 142 may be selectively movable relative to the bone anchoring portion 141 in one or more selected directions to facilitate insertion of the transverse rod 16 in the receiving portion 142, or may be fixed relative to the bone anchoring portion. Preferably, the rod-receiving portion 142 locks in a selected orientation relative to the bone anchoring portion 141 when the locking mechanism 145 is inserted. Each connector 18 includes a transverse rod-receiving portion and a spinal rod-receiving portion for coupling the spinal rod 12 to the transverse rod 16. As shown, the spinal rod 12 extends in a direction substantially parallel to the spinal column, while the transverse rod 16 extends substantially transverse to the spinal rod 12. A locking mechanism, such as a screw cap 185, in the connector 18 locks the transverse rod 16 and spinal rod 12 in a selected relationship relative to each other and the connector 18.

According to an alternate embodiment, the spinal rod 12 or other spinal corrective element may be directly coupled to the bone anchors 14, which would then include rod-receiving portions extending substantially parallel to the spinal column 20 to receive the spinal rod 12.

The spinal rod 12 may have a circular cross-section, a polygonal-shaped cross-section or a cross-section with any other suitable shape. For a single-rod spondylolistheses correction system 10, as shown in FIGS. 1A and 1B, the single spinal rod 12 preferably has square cross-section to provide torsional correction and stability during correction of the spondylolistheses defect.

As shown in FIG. 2, a spondylolistheses correction system 10′ may comprise two spinal rods 12A and 12B straddling the vertebrae 20 and connected directly to the bone anchors 14A and 14B or via intermediate connectors 18 and transverse rods 16.

The spinal corrective device 12 applies corrective forces, which are transferred to the spine via the bone anchors 14, to reduce the spondylolistheses defect. Due to its visco-elastic properties, the spine will be corrected by the corrective forces of the spondylolistheses correction system 10. The spinal corrective device 12 preferably changes shape after attachment to the spine via the bone anchors 14 to create the corrective forces for correcting the spondylolistheses defect.

Each spinal rod 12 or other spinal corrective device in the spondylolistheses correction system 10 is preferably formed of or includes a shape memory material, which may be a shape memory polymer or alloy, such as nitinol (a nickel-titanium alloy), to allow for the change in shape that creates the corrective forces. Shape memory materials are characterized by an ability to restore the material to a pre-selected shape of a particular state after plastic deformation. For example, in an austenitic state, the shape memory material is stiff, rigid and has a set, pre-selected shape. In a martensitic state, the shape memory material becomes flexible and deformable and may have any of a variety of shapes. The microstructure of the material in the martensitic state is characterized by “self-accommodating twins”, having a zigzag arrangement, which allow for deformation of the material shape by de-twinning. The shape memory material forming the rod 12 converts to a stiff, high-strength structure in an austenitic state. In a martensitic state, the rod material becomes more flexible and can be more easily bent into a variety of different shapes.

Generally, shape memory materials are induced to transition between the rigid austenitic state and the flexible martensitic state by changing the temperature, pressure, stress, chemistry and/or another parameter of the shape memory material.

In the illustrative embodiment, the shape memory material of a spinal corrective device used for correcting spondylolistheses transitions between martensitic and austenitic states by changing the temperature of the material. The spinal rod 12 may be cooled to make the material flexible in a martensite state, and subsequently heated to return the material to the original shape with the austenitic structure. For example, in one embodiment, a material in the martensitic state returns to the pre-selected shape of the austenitic state by changing the temperature of the material, usually by heating the material, above a selected temperature. The temperature at which a shape memory material starts transforming to austenite is known as the “austenite start temperature.” Further heating increases the temperature of the shape memory material to induce a complete transformation to the austenitic state. The temperature at which a shape memory material finishes transforming to austenite is known as the “austenite finish temperature.”

A shape memory material can transition to the martensitic state to allow deformation and shaping of the spinal corrective device by changing the temperature of the material below a selected temperature (i.e., cooling the material). The temperature at which a shape memory material begins transformation to the martensite state is known as the “martensite start temperature”. Further cooling decreases the temperature of the shape memory material to induce a complete transformation to the martensite state. The temperature at which a shape memory material finishes transformation to the martensite state is known as the “martensite finish temperature.”

In the illustrative embodiment, the martensite start temperature is preferably between about 15 and about 20 degrees Celsius less than the austenite start temperature, which is preferably between about 0 degrees Celsius and about 10 degrees Celsius. The shape memory material preferably has an austenite finish temperature that is below body temperature (37 degrees Celsius), for example, 32 degrees Celsius, such that when the shape memory material reaches equilibrium with the environment within the body, the shape memory material will exist in the fully austenitic state. At room temperature, the spinal corrective device is preferably already in transition to the austenitic state to put force on the spine. One skilled in the art will recognize that the transition temperatures between and within the martensite and austenite states may be selected to be any suitable temperature, depending on the composition of the shape memory material and/or the manufacturing process used to produce the shape memory material.

When used in spondylolistheses corrective surgery, the spinal rod 12 of the spondylolistheses correction system 10 in the austenitic state preferably has the curved lordotic shape of an expected corrected spine. The pre-selected shape in the austenitic shape may be determined by a surgeon prior to surgery and formed by heat-working the material to the selected shape or using other means known in the art.

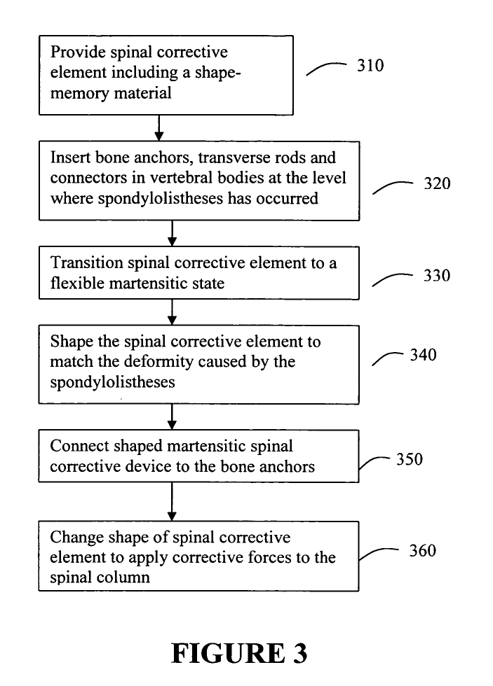

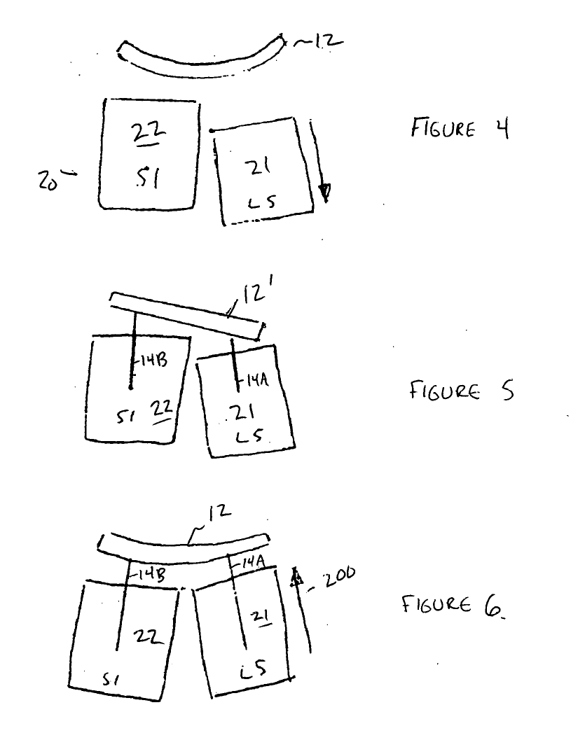

FIG. 3 is a flow chart illustrating the steps involved in correcting a deformity caused by spondylolistheses, when one vertebra is displaced forward relative to other vertebral bodies in the spinal column, using a spondylolistheses correction system 10 of an illustrative embodiment of the invention. FIGS. 4-6 illustrate the state of the spinal column 20 and a spinal correction device, such as a spinal rod 12, of a spondylolistheses correction system during the steps shown in FIG. 3.

In a first step 310, at least one spinal corrective element, such as a spinal rod 12, formed at least partially of a shape-memory material, such as nitinol, is provided. The spinal rod 12 may be provided in the austenitic state, where the rod has the curved lordotic shape of an expected corrected spine, as shown in FIG. 4. The initial spinal corrective element may have any of a variety of lengths and curvatures to accommodate particular anatomical variations of the individual patient.

In step 320, bone anchors 14, such as polyaxial bone screws, transverse rods 16 and connectors 18 are inserted in vertebral bodies at the level where spondylolistheses has occurred. A first pair of bone anchors 14A is anchored to a forwardly displaced vertebra 21, while at least one other pair of bone anchors is anchored to a non-deformed vertebra 22, to which the forwardly displaced vertebra is to be brought into alignment. Several pairs of bone anchors may be used, if necessary.

The spinal corrective element, such as the spinal rod 12, transitions in step 330 from a stiff austenitic state to a flexible martensitic state, for example, by cooling the shape memory material in the spinal corrective element. The step of cooling or otherwise transitioning the shape memory material to a martensitic state can occur before or after the bone anchors are inserted in step 320. For a nitinol material, the spinal rod 12 is preferable cooled to a temperature of −30 degrees Celsius, below the martensite finish temperature.

The spinal corrective element may be cooled to a martensitic state using any suitable means known in the art. For example, the spinal corrective element may be inserted in a freezer or other reduced-temperature environment to cool the shape memory material to a martensitic state. Alternatively, an external cooling device may be used to transition the shape memory material to a martensitic state. For example, a cold gas, such as liquid nitrogen or dry ice (CO₂), or other coolant may be applied directly or indirectly to the shape memory material to cool the material and transition the material to the flexible, deformable martensitic state.

While the material is in the martensitic state, the surgeon shapes the spinal corrective element, in step 340, to match the deformity caused by the spondylolistheses. The spinal corrective element may be shaped manually, using benders, or using any suitable instrument. Shaping the flexible spinal corrective element to match the deformity allows the spinal corrective element to match and fit into receiving portions on the connectors 18 and/or bone anchors 14 already connected to the spine.

In one embodiment, a phantom spinal rod may be used in step 340 to shape a martensitic spinal rod to match a spondylolistheses deformity. The phantom spinal rod is flexible and may be bent and/or twisted to fit into the receiving-portions of the connectors 18 or bone anchors 14 to determine the shape of the deformed spine. The cooled spinal rod, which is flexible and deformable while below the martensite start temperature, and is preferably below the martensite finish temperature, is bent and/or torded to match the phantom spinal rod, and thus the spondylolistheses deformity.

After shaping the spinal corrective element in step 340, the shaped martensitic spinal corrective device, such as a shaped spinal rod 12′ as shown in FIG. 5, is inserted in a spinal corrective device-receiving portion in the bone anchors 14 or connectors 18 to connect the shaped martensitic spinal corrective device to the bone anchors 14 in step 350, as shown in FIG. 5. Approximators may be used, if needed, to approximate the spinal corrective element into the receiving portion. The shaped martensitic spinal rod 12′ or other spinal corrective element is then fixed to the bone anchors, either directly or indirectly, to be able to withstand a selected amount of corrective force for realigning the vertebrae.

In step 360, the shaped spinal corrective element 12′ changes shape to apply corrective forces to the spinal column. In the illustrative embodiment, the surgeon effects the change in shape by transitioning the spinal corrective element to the austenitic state, as shown in FIG. 6, which causes the spinal corrective element, illustrated as spinal rod 12, to revert to the original shape of the curved lordotic shape of an expected corrected spine. Preferably, the surgeon transitions the spinal rod to the austenitic state by heating the spinal rod to or past the austenitic start temperature, and preferably past the austenite finish temperature.

As shown in FIG. 6, because the spinal corrective device, illustrated as spinal rod 12, is fixed to a deformed vertebral body 21, the movement of the spinal corrective device back to the original shape of the austenitic state applies corrective forces to pull the deformed vertebral body 21 back into alignment with the spinal column 20, in the direction indicated by arrow 200.

Any suitable means may be used to transition the spinal corrective element to the austenitic state. For example, a heater for heating a shape memory material in a spinal corrective element may employ a heated liquid that circulates near or in contact with the shape memory material to raise the temperature of the shape memory material into the austenitic state. Alternatively, a heater may employ induction heating, resistance heating, electromagnetic radiation heating and/or any other suitable means for increasing the temperature of the shape memory material. According to one embodiment, body heat may be used to partially or fully heat the shape memory material to the austenitic state.

Preferably, the shape memory material is heated above the austenitic final temperature and above body temperature to create sufficient force to move the spine into the corrected configuration, while keeping the temperature sufficiently low (i.e., equal to or below about 40 degrees Celsius) to prevent burns to the body.

As shown in FIG. 6, the corrective force applied to the spine is applied parallel to and away from the axis of the anchoring portion 141 of the bone anchors 14, against the locking mechanism 145 of the bone anchors 14, thereby pulling the displaced vertebra in a rearward direction.

The correctional force applied to the spine to correct the deformity caused by spondylolistheses is preferably applied evenly and spread across the vertebral bodies, in contrast to prior spondylolistheses correction system, which apply an uneven, concentrated force to a displaced vertebral body to pull the displaced vertebral body back into alignment.

In one embodiment of the invention, isolated segments of the spinal corrective device may be controllably transitioned between the martensitic state and the austenitic state at a time. The spinal corrective element may have insulation between different segments of the element to prevent heat from transmitting from one segment to another to facilitate segmental correction. In another embodiment, the entire spinal corrective element may transmit heat, so that heating a portion of the spinal corrective element transmits heat to other portions of the spinal corrective element to transition the entire spinal corrective element to an austenitic state.

According to one embodiment, the vertebrae are fully reduced during the surgical procedure, i.e., the temperature of the shape memory material increases past the austenite final temperature during the surgical procedure, so that no additional transition occurs after surgery.

According to another embodiment, the vertebrae are not fully reduced during the surgical procedure, to allow for post-operative correction as the spinal corrective device continues to heat up to body temperature after surgery. In this manner the spondylolistheses defect may be reduced fully over time.

In addition, the method of correcting a spondylolistheses defect using a shape memory material may employ in vivo cooling of the spinal corrective element to control the corrective forces applied to the spine. For example, if excessive or damaging force is applied to the spine during transition to the austenitic state, a cooling device may selectively and controllably cool the shape memory material to remove some of the corrective force and loosen the spine. Then, heating may be subsequently controllably applied to reapply the corrective forces to the spine. Alternatively, after releasing the spine by in vivo cooling, additional anchoring devices may be inserted and connected to the spinal corrective element to spread the load of the corrective forces over a greater area to reduce damage.

The spinal correction system 10 may also include a feedback mechanism to allow for control of the heating and/or cooling and/or other transition-inducing parameter. For example, a sensor may measure the temperature of the shape memory material in the spinal corrective element, which may be used to adjust the heating and/or cooling of the material to control the transition between states.

The system of method of correcting spondylolistheses in a patient using a shape memory material provides significant advantages over prior spondylolistheses correction systems. The forces required to move the displaced vertebra can be controlled are focused closer to the spine, and spread across the implants, potentially reducing the overload to the spine or implants. In addition, simpler implementation can be used. The system and method allow for inter-operative correction and post-operative correction over time to give a better clinical result.

The present invention has been described relative to an illustrative embodiment and application. Since certain changes may be made in the above constructions without departing from the scope of the invention, it is intended that all matter contained in the above description or shown in the accompanying drawings be interpreted as illustrative and not in a limiting sense.

It is also to be understood that the following claims are to cover all generic and specific features of the invention described herein, and all statements of the scope of the invention which, as a matter of language, might be said to fall therebetween.

1. A method of correcting spondylolistheses in a spinal column in a patient, comprising the steps of:
anchoring a first bone anchor to a first vertebra and a second bone anchor to a second vertebra that is displaced forward of the first vertebra due to the spondylolistheses;
connecting the first bone anchor to the second bone anchor using a spinal corrective element; and
changing the shape of the spinal corrective element to apply a corrective force to reduce the spondylolistheses.

2. The method of claim 1, wherein the corrective force pulls the second vertebra back into alignment with the first vertebra, thereby correcting the spondylolistheses.

3. The method of claim 1, wherein the spinal corrective element includes a shape memory material.

4. The method of claim 3, wherein the shape memory material comprises nitinol.

5. The method of claim 3, wherein the step of changing the shape of the spinal corrective element comprises heating the spinal corrective element to transition the shape memory material in the spinal corrective element to an austenitic shape in which the spinal corrective element has a curved lordotic shape of an expected corrected spine.

6. The method of claim 3, further comprising the step of transitioning the spinal corrective element to a flexible martensitic state while attached to the vertebrae to allow reshaping of the spinal corrective element while attached to the vertebrae.

7. The method of claim 1, wherein the step of connecting the first bone anchor and the second bone anchor comprises the steps of:
cooling a spinal corrective element formed of a shape-memory material to a martensitic state; and
bending the spinal corrective element to match a deformity caused by the spondylolistheses; and
inserting a first portion of the spinal corrective element in a receiving portion connected to the first bone anchor and a second portion of the spinal corrective element in a receiving portion connected to the second bone anchor.

8. The method of claim 1, wherein at least a portion of the step of changing the shape of the spinal corrective element occurs post-surgery.

9. A method of correcting spondylolistheses in a patient, comprising the steps of:
shaping a spinal corrective element to match a deformity caused by spondylolisthesis; and
fixing the shaped spinal corrective element to a first vertebra and a second vertebra that is displaced from the first vertebra in a forward direction due to the spondylolistheses.

10. The method of claim 9, wherein the spinal corrective element includes a shape memory material.

11. The method of claim 10, wherein the step of shaping the spinal corrective element to match the deformity comprises cooling the shape memory material to a martensitic state and bending the spinal corrective element to match the deformity.

12. The method of claim 9, further comprising the step of:
adjusting the shape of the spinal corrective element to apply a corrective force to the spine, pulling the second vertebra into alignment with the first vertebra.

13. The method of claim 12, wherein the spinal corrective element includes a shape memory material having a curved lordotic shape of an expected corrected spine when in an austenitic state.

14. The method of claim 13, wherein the step of adjusting the shape of the spinal corrective element to apply the corrective force comprises heating the shape memory material to the austenitic shape, causing the spinal corrective element to have the curved lordotic shape while fixed to the first and second vertebrae.

15. A method of correcting spondylolistheses in a patient, comprising the steps of:
inserting a spinal corrective element in receiving portions connected to bone anchors, wherein the spinal correction element includes a shape memory material and is shaped to match a deformity caused by the spondylolistheses; and
transitioning the shape memory material from a martensitic state to an austenitic state to reduce the deformity caused by the spondylolistheses.

16. The method of claim 16, wherein spinal fixation element pulls a displaced vertebra back into alignment with adjacent vertebra.

17. The method of claim 15, wherein the spinal correction element in the austenitic state has a curved lordotic shape of an expected corrected spine.

18. The method of claim 15, wherein the spinal correction element in the martensitic state has a shape that matches the deformity caused by the spondylolistheses.

19. The method of claim 15, wherein the spinal correction element is fixed to the fix rod to spine while in martensitic state.

20. The method of claim 15, wherein the spinal correction element pulls against locking mechanisms in the receiving portions during transition.

21. The method of claim 16, wherein the spinal correction element pulls the forwardly displaced vertebra in a direction parallel to and away from a longitudinal axis of a bone anchor.

22. A method of correcting spondylolistheses in a patient, comprising the steps of:
fixing a spinal corrective element to a first vertebra and a second vertebra that is forwardly displaced relative to the first vertebra due to the spondylolistheses; and
applying a substantially even corrective force distributed across the vertebrae to pull the second vertebra into alignment with the first vertebra.

23. The method of claim 22, wherein the step of applying the substantially even corrective force comprises changing a shape of the spinal corrective element from a first shape matching a spondylolistheses deformity to a curved lordotic shape of an expected corrected spine.

24. The method of claim 23, wherein the step of changing a shape comprises heating the spinal corrective element to transition a shape memory material in the spinal corrective element from a martensitic state to an austenitic state.

25. A spondylolistheses correction system for correcting spondylolistheses in a spinal column in a patient, comprising:
a first set of bone anchors connected to a first vertebra;
a second set of bone anchors connected to a second vertebra that is displaced forward of the first vertebra due to the spondylolistheses; and
a spinal corrective element including a shape memory material connecting the first set of bone anchors and the second set of bone anchors.

26. The spondylolistheses correction system of claim 25, further comprising:
a first transverse rod extending between the first set of bone anchors;
a first connector for connecting the spinal corrective element to the first transverse rod;
a second transverse rod extending between the second set of bone anchors; and
a second connector for connecting the spinal corrective element to the second transverse rod.

27. The spondylolistheses correction system of claim 25, wherein the spinal corrective element has a square-shaped cross section.

28. The spondylolistheses correction system of claim 25, wherein the spinal corrective element has a curved lordotic shape of an expected corrected spine when in an austenitic state and is flexible in a martensitic state to match a spondylolistheses deformity.

29. The spondylolistheses correction system of claim 28, further comprising a temperature control element for changing the temperature of the spinal corrective element to transition the shape memory material between the martensitic state and the austenitic state.

30. The spondylolistheses correction system of claim 25, further comprising a second spinal corrective element including a shape memory material and connecting the first set of bone anchors and the second set of bone anchors.
